(19) United States
(12) Patent Application Publication
(10) Pub. No.: US 2010/0310656 A1
(43) Pub. Date: Dec. 9, 2010

(54) Immunotherapy and prevention of autoimmune hepatitis

(75) Inventors: Aldar S. Bourinbaiar, Rockville, MD (US); Vichai Jirathitikal, Chachoengsao (TH)

Correspondence Address: Baker Donelson Bearman, Caldwell & Berkowitz, PC, 920 Massachusetts Ave, NW, Suite 900, Washington, DC, 20001, US

(73) Assignee: Immunitor USA, Inc., College Park, MD (US)

(21) Appl. No.: 12/662,663

(22) Filed: Apr. 27, 2010

Related U.S. Application Data
(60) Provisional application No. 61/172,829, filed on Apr. 27, 2009.
(60) Provisional application No. 61/240,491, filed on Sep. 8, 2009.

(51) Int. Cl. A61K 39/29; C07K 14/00; A61K 9/00; A61K 39/12; A61K 35/12; A61K 39/00; A61P 31/12; A61P 31/00

(52) U.S. Cl. 424/484; 530/350; 530/380; 424/204.1; 424/225.1; 424/93.7; 424/93.6; 424/184.1

(57) ABSTRACT

The invention is within the field of immunology and microbiology, more specifically the field of virology and is related to immunotherapy and prophylaxis of hepatitis and autoimmune diseases. The composition useful for these purposes is disclosed, including the methods of making and using said composition.

### FIELD OF THE INVENTION

The present invention relates to the immunotherapy and prophylaxis of hepatitis and other autoimmune diseases. In particular the invention relates to a composition, methods of making said composition, and methods of use.

### BACKGROUND OF THE INVENTION

Hepatitis can be of viral origin. Hepatitis of viral origin is associated with five viruses found in the liver and other cells in humans and other host species, these are usually abbreviated by letters from A to E. Other new hepatitis viruses were discovered recently. These viruses are described briefly hereinafter to provide the background to the invention.

Hepatitis A virus (HAV)—is a small, spherical, and exceptionally resistant RNA-virus. It is transmitted preferentially by the fecal-oral route and replicates exclusively in the liver. Infection is usually self limiting, yet individual cases may also take a protracted and even relapsing course. True chronic infections, however, are not observed. HAV has a world-wide distribution. In countries where inadequate sanitary conditions prevail, the virus persists in the environment and almost 100% of the population acquires infection in childhood. With increasing age infections become more and more clinically manifest and at and beyond of adolescence more than 80% of patients develop icteric, in some cases even fulminant and fatal hepatitis. Preventive measures rely on strict personal and alimentary hygiene as well as on vaccination with killed hepatitis A vaccines. These vaccines are safe, highly immunogenetic and induce long lasting protection against hepatitis A. Specific antiviral therapy is not yet available.

Hepatitis B virus (HBV), a DNA virus of a large hepadnaviridae family, affects an estimated 350 million individuals and is the leading cause of chronic liver disease and hepatocellular carcinoma worldwide. Approximately 500,000 deaths per year are attributed to HBV. A plasma-derived hepatitis-B vaccine has become widely available since 1981. In 1987 the FDA licensed the first genetically engineered hepatitis B vaccine—Recombivax HB manufactured by Merck Sharp & Dohme. While vaccination programs have reduced HBV incidence in many countries, it still remains a major public health problem, especially in Asia and Africa.

Hepatitis C virus (HCV) is an enveloped RNA virus in the Flaviviridae family which appears to have a narrow host range. Humans and chimpanzees are the only known species susceptible to infection, with both species developing similar disease. HCV is another global public health problem, infecting estimated 170 million people. The WHO estimates that 3 to 4 million persons are newly infected with HCV each year. Approximately 85% of acute infections progress to chronic persistence of HCV and about 25% of these chronically infected individuals develop fatal liver diseases. Currently, there is no prophylactic vaccine to prevent the disease and no specific antiviral drug controlling HCV replication.

Hepatitis D virus (HDV) is a subviral satellite with hepatitis B virus (HBV) as its natural helper virus. After entry into hepatocytes, it utilizes host cellular machinery to replicate by a double-rolling-circle mechanism. HDV is most often transmitted by contact with contaminated blood and body fluid, similar to HBV infection. Approximately 5% of the global HBV carriers are co-infected with HDV, leading to a total of 10-15 million HDV carriers worldwide. HDV infection can occur concurrently with HBV infection (co-infection) or in a patient with established HBV infection (superinfection). Vaccination strategies tested in the woodchuck model induced specific B- and T-cell responses but failed to protect from HDV infection. Treatment of HDV is also problematic and no effective therapy exists that could eliminate this virus.

The hepatitis E virus (HEV) is a small RNA virus and the etiological agent for hepatitis E, a form of acute viral hepatitis. HEV is the sole member of the genus Hepevirus in the family of Hepeviridae. HEV is a major cause of outbreaks and sporadic cases of viral hepatitis in tropical and subtropical countries but is infrequent in industrialized countries. The overall death rate among young adults and pregnant women is 0.5-3% and 15-20%, respectively. The virus has a feco-oral transmission cycle and is transmitted through environmental contamination, mainly through drinking water. Recent studies on the isolation of HEV-like viruses from animal species also suggest zoonotic transfer of the virus from animals such as swine. A cell culture system for the propagation of the virus has been discovered and preliminary, promising trials of prophylactic HEV vaccine have been described.

Hepatitis G virus (HGV), was discovered in the late 1990s. The prevalence in the general population is high—about 2%. HGV is a single stranded RNA virus which belongs to the flavivirus family. HGV is distinct from HCV. The newly discovered flaviviris agents, GBV-A and GBV-B are also distinct from HGV, while GBV-C represents an isolate of HGV. A classification of GBV-C/HGV strains into at least three genotypes (West Africa, Europe/North America, Asia) has been proposed. The structure of the HGV genome resembles that of HCV. HGV replicates in peripheral blood cells, while replication in liver cells has not been observed till date. Analysis of many studies dealing with HGV indicate that this virus is the lymphotropic. HGV or GBV-C has been ascertained to influence course and prognosis in the HIV-infected patient. Until now, the frequent presence of GBV-C in coinfections, hematological diseases, and biliary pathology gives no grounds to determine it as a harmless virus without pathological significance.

A number of new hepatitis viruses, such as transfusion transmissible circular DNA virus (TTV) and SEN (SEN-V) a blood-borne, single-stranded, non-enveloped DNA virus, were discovered recently. There are no prophylactic or therapeutic vaccines exist against these viruses.

With the exception of hepatitis A, B, and E no effective prophylactic vaccines against other viruses have been developed and there is an unmet need for such vaccines. Furthermore, no therapeutic vaccines against any of above hepatitis viruses have been developed, emphasizing the critical need for such vaccines.

In addition to viral hepatitis there are other forms of hepatitis which have similar clinical symptoms such as for example, alcohol-induced hepatitis or iatrogenic hepatitis, i.e., induced by hepatotoxic drugs or substances. While hepatitis is often triggered by unrelated viruses or other factors they are nevertheless only the triggers of the disease. The main cause for hepatitis is a dysfunctional immune reaction which attacks the liver and causes inflammation.

Proteins, polypeptides, and peptides commonly referred to as antigens are typically administered via injection or other route to avoid the digestive degradation. The oral delivery of antigens for therapeutic or prophylactic use is a many decades' long challenge for pharmaceutical and vaccine industry yet to be solved. The unsatisfactory state of the art in oral vaccines is described by us extensively in a review article (Silin et al., Oral vaccination: where we are? Expert Opinion Drug Delivery 2007; 4:1-18). Applicant's prior clinical studies have shown that heat-inactivated hepatitis antigens are effective and safe when administered orally to hepatitis patients (Batdelger et al., Open label trial of therapeutic hepatitis B vaccine V-5 Immunitor (V5) delivered by oral route. Lett Drug Design Discovery 2007; 4:540-44; Batdelger et al., Open label trial of therapeutic immunization with oral V-5 Immunitor (V5) vaccine in patients with chronic hepatitis C. Vaccine 2008; 26:2733-2737).

Hydrolyzed blood products have been in clinical use since 1960's. Examples of such products are Hydroprot, Aminokrovin, Infusamin, Fibrinosol, etc., which are made from human or animal blood by adding acid and glucose. Their exclusive mode of delivery is intravenous route or rectal infusion, as these products are not effective when given orally since upon entering the stomach they lose biological activity due to digestive process. Furthermore, these hydrolyzates result from acid hydrolysis process and are essentially composed of amino acids. Thus, these products are devoid of any antigenicity or immunogenicity that is associated with longer sequences of amino acids such as peptides and polypeptides.

It is commonly held that hepatitis antigens used in vaccine composition are not stable or biologically active under low or high pH and exposing antigens to these conditions is not advisable. However some hepatitis viruses adapted to grow in the gastrointestinal tract, for example hepatitis A and E, remained quite infectious at low pH (Scholz et al., Acid stability of hepatitis A virus. J Gen Virol. 1989 September; 70 (Pt 9):2481-5), mainly because they are non-enveloped viruses, which are generally much “tougher” in hostile environment than enveloped viruses. However other viruses are much more sensitive to both low pH and heat treatment and lose antigenicity at milder conditions (see for example Rombaut et al., Thermal inactivation of oral polio vaccine: contribution of RNA and protein inactivation. J Virol 1994 October; 68(10):6454-7).

Thus, the current consensus prevailing in the vaccine field holds that the hydrolysis results in loss of antigenicity, i.e., loss of biological activity, which renders a vaccine, made from hydrolyzed antigens, totally useless, especially when antigens also have been exposed to high temperature environment (see for example Nath et al., Stability of the recombinant hepatitis B core antigen. J Clin Microbiol 1992 June; 30(6):1617-9; Nuanualsuwan and Cliver. Inactivation of picornaviruses and caliciviruses; Part 2: Inactivation. That J Veterinary Medicine 30 Sep. 2003; Vol. 33 No. 3, and references therein).

The current standard of care for hepatitis C is administration of interferon alfa-2a (Roferon-A) and interferon alfa-2b (Intron-A) or pegylated interferons (PEG-Intron, Pegasys) alone or in combination with ribavirin (Copegus, Rebetol). Treatment response is dependent on viral subtype, with 76 to 80% of those with genotypes 2 and 3, but only approximately 40% with genotype 1 or 4 achieving a sustained virologic response. The most prescribed treatment for hepatitis B is administration of interferon alpha and its pegylated version Pegasys. Glaxo-Wellcome's product lamivudine (Epivir) was launched in 1998. A second hepatitis drug, adefovir dipivoxil (Hepsera, Gilead Sciences), was approved by the FDA in September 2002. Entecavir (Baraclude) from Bristol-Myers Squibb was approved in March 2005. Recently approved telbivudine (Tyzeka) is another nucleoside analogue and was developed by Idenix. These drugs act as virostatic agents by reducing the amount of replicating virus, however the virus generally returns once the treatment is discontinued. In addition, most of these drugs have significant adverse or toxic effects. Many patients drop out or decline the treatment because of side effects. Viral resistance is another drawback of long-term antiviral chemotherapy. Lamivudine monotherapy is associated with higher resistance (year 1, 10-27%; year 2, 37-48%; year 4, 60-65%) than adefovir (year 1, 0%; year 2, 3%; year 5, 29%) or telbivudine (year 1, 3-4%; year 2, 9-22%). Entecavir resistance is rare in naive individuals (year 4, <1%), but increases over time to 43% in lamivudine-resistant patients.

It is thus clear that better treatment modalities are needed to improve clinical outcome in hepatitis patients. One approach that can overcome current deficiencies in treatment of hepatitis is disclosed in the present invention as described hereinafter.

### SUMMARY OF THE INVENTION

The present invention departs from generally held beliefs that sophisticated compositions are necessary to overcome the lack of progress in developing effective immunotherapy and prophylaxis of hepatitis. The present invention features a surprising discovery that hydrolyzed hepatitis virus antigens are not inferior in their clinical efficacy than regular heat-inactivated viral antigens. Thus, this composition can be regarded as a vaccine for treatment or prevention of a viral infection hepatitis or autoimmune hapatitis. Another feature that enhances the attractiveness of the instant discovery is that the claimed composition is effective in oral dosage form. In order to be biologically available, an antigen of the instant invention is embedded or entrapped within pharmaceutically acceptable matrix. Furthermore, no immune adjuvant is required to produce the desired effect when the composition is administered orally.

In one aspect, this invention comprises a solid oral composition comprising a therapeutically effective amount of a partially hydrolyzed antigen, wherein said partially hydrolyzed antigen is derived from a body fluid of a donor infected with a hepatitis virus. The solid oral composition is conveniently made into a tablet or pill. The body fluid can comprise a hepatitis virus or hepatitis virus antigen(s). This composition further comprises an alloantigen or xenoantigen that are embedded into a matrix such a metal salt. In another more general aspect of the invention the instant composition comprises an acid-hydrolyzed antigen, an alloantigen, or combination thereof embedded in a metal matrix.

Hydrolyzed antigens can be used as an immunotherapy or as a prophylactic preparation for preventing hepatitis infections such as a hepatitis C virus (HCV), hepatitis B virus (HBV), hepatitis G virus (HGV), hepatitis delta virus (HDV), hepatitis A virus (HAV), and other known and yet to be discovered hepatitis viruses. The invention is equally applicable to viruses belonging to hepatitis virus families including flaviviruses. Specifically, this invention relates to a composition, and methods of making and using said composition.

A second aspect of the present invention is a composition comprising both an alloantigen and a hepatitis virus antigen. This is another feature that contributes to the surprising aspect of the present invention. A preferred embodiment of this feature is a solid composition comprising an partially acid-hydrolyzed alloantigen embedded in an orally available metal salt matrix, wherein the alloantigen or xenoantigen is a human alloantigen embedded in a divalent metal salt matrix and preferably heat-treated. The preferred administration of such a composition is oral.

Thus, a preferred feature of the present invention is that the composition is preferably in solid oral form such as a tablet or pill.

Yet another aspect of the present invention is a process of making the instant composition which in general comprises partially hydrolyzing a hepatitis virus, a fragment of a hepatitis virus, or an alloantigen, embedding the hydrolysate into a solid matrix, and formulating the matrix into a composition for oral administration.

In another aspect of this invention hydrolyzed antigens are heat treated to render the instant composition safe and kill any remaining, un-hydrolyzed, adventitious viruses which may become a source of infection. While this step is important, it is not critical, since the process of hydrolysis and embedding is accomplished at substantially elevated temperature, at least 56° C., preferably higher than 80° C., and for prolonged periods of time such as longer than 2 hours.

In another aspect of the invention, the composition is administered without an immune adjuvant.

While it is contemplated that the composition is derived from body organs or body fluids like the whole blood, or plasma or serum, the present invention also anticipates a recombinant virus and virus antigens such as those well known in the art and medical literature references. These recombinant viruses or parts thereof comprise naturally occurring sequences of amino acids or nucleic acids which constitute such antigens, derivatives of naturally occurring sequences that are substantially similar to a naturally occurring sequence, or sequences that mimic them either in linear or tertiary forms of, and chimeric sequences that may have more than one hepatitis virus, such as for example HBV and HCV within one composition.

These and other permutations of various forms of a vaccine are contemplated without restriction and well known in the art as described for example by Silin et al., (Silin et al., Oral vaccination: where we are? Expert Opinion Drug Delivery 2007; 4:1-18), the contents of which is a part of this disclosure in its entirety.

A further aspect of the present invention embodies a method of treating a patient comprising the step of administering to the patient a therapeutically effective amount of partially hydrolyzed hepatitis virus-infected cell, a hepatitis virus, or fragment thereof. In addition to treating a patient infected with a hepatitis virus or the present invention embodies a method of administering said vaccine prophylactically in order to reduce the likelihood or severity of eventual hepatitis infection.

In an additional embodiment, the invention is directed to a method for eliciting a cellular immune response in a vertebrate subject comprising administering to a vertebrate subject a therapeutically effective amount of a selected macromolecule embedded into metal matrix of the invention.

### DETAILED DESCRIPTION OF THE INVENTION

### Overview

As opposed to prior attempts the instant invention addresses not the triggers but the true cause of hepatitis, which is an autoimmune reaction against infected liver. Thus, this invention, in general, addresses treatment and prevention of autoimmune diseases. Therefore, the instant invention addresses an issue that has seldom been investigated. As such this invention is set against obvious dogmas that have prevailed too long in the field and that need to be overcome in order to give a place to progress.

More specifically, Applicants here disclose that when HBV or HCV antigens are partially hydrolyzed prior to oral administration, they were surprisingly more effective and safe than heat-inactivated antigens that have not been subjected to hydrolysis.

### DEFINITIONS

As defined herein hepatitis is injury to the liver characterized by the presence of inflammatory cells in the tissue of the organ, which damage the liver. Regarded from this viewpoint, hepatitis is an autoimmune disease which arises from an overactive immune response of the body against own tissue. In other words, the immune system attacks its own cells.

The damage of the liver ensuing from HAV infection most likely does not stem directly from virus replication but is the result of an interaction of cell mediated virus-specific immunity with infected hepatocytes.

As used herein the term “hepatitis virus antigen” refers to any antigenic material derived from a hepatitis virus, either native or recombinant, which may be used to produce beneficial clinical effect among those in need thereof, such as humans. As used herein the term “partially hydrolyzed antigen” refers to an antigen resulting from hydrolysis to smaller fragments than in original native form. Hydrolysis is carried out by acid or alkali, more specifically referring to acid-mediated hydrolysis. Other forms of hydrolysis such as an enzyme hydrolysis are referred to as a “digestion”.

Both terms “alloantigen” and “xenoantigen” can be alternatively named as a “non-self antigen” and used equally to mean each of the former terms separately. An alloantigen is an antigen presented in allelic forms as encoded at the same gene locus in different individuals of the same species.

Alloantigen can be any protein, peptide, amino acid, or nucleic acid that serves as an alloantigen. For example, alloantigen is presented to the immune system of a host during alloimmunization—a situation, well known to transplantation immunologists, when foreign tissue or cells are transplanted from one individual to another of the same species. Similar situation arose during xenotransplantation when xenoantigen is transplanted from the individual of one species to an individual from another species.

The term “adjuvant” as used by vaccine experts refers to any compound which, when injected together with an antigen, non-specifically enhances the immune response to that antigen. While the preferred mode of instant administration is without and adjuvant, one skilled in the art, can administer the composition with an immune adjuvant known in the art. Of particular advantage are those that favorably induce immune tolerance and yet at same time produce beneficial clinical effect and capable of clearing the infection.

A “patient” refers to a host such a human capable of being infected with hepatitis virus. A patient may or may not be infected with hepatitis virus. Examples of patients are humans, chimpanzees, ducks, pigs, dogs, cats, woodchucks and other species capable of being infected with hepatitis virus.

By definition the term “antigen” means a molecule which contains one or more epitopes capable of stimulating a host's immune system to make a cellular immune response when the antigen is presented in accordance with the present invention, or a humoral antibody response. The capacity to induce the immune response is understood as “immunogenicity”.

The term “therapeutically effective amount” implies an amount that will be included in the composition which will cause the subject to produce a sufficient response, in order to prevent, reduce, eliminate or diagnose symptoms. The exact amount necessary will vary, depending on the subject being treated; the age and general condition of the subject to be treated; the severity of the condition being treated; in the case of an immunological response, the capacity of the subject's immune system to synthesize antibodies; the degree of protection desired and the particular antigen selected and its mode of administration, among other factors. An appropriate effective amount can be readily determined by one of skill in the art. Thus, a “therapeutically effective amount” will fall in a relatively broad range that can be determined through routine experimentation such as a clinical trial.

Unless explicitly stated, reference to terms such as “a” or “an” is not limited to one. For example, “a vaccine” does not exclude the multitude of vaccines. Occasionally phrases such as one or more are used to highlight the possible presence of a plurality.

### DETAILED DESCRIPTION

In the course of experimentations it has been surprisingly discovered that when hepatitis viruses, such as those found in the blood of hepatitis virus carriers, are partially hydrolyzed, they acquire biological activity in terms of inducing more favorable clinical outcome when they are administered to hepatitis sufferers or as means of prophylaxis of infection.

The partially hydrolysis can be easily achieved by acid treatment as the preferred mode of hydrolysis. The pH range is preferably below 6 and more preferably between 0.5 and 5.5, more preferably between 1 and 4, more preferably between 1.0 and 3.0, and even more preferably between 1.0 and 2.0. While these ranges are given as optimal ranges, they will not prevent one skilled in the art to determine the optimal range, which remains within the scope of the present invention. The essential embodiment of the invention is that partially hydrolysis is performed in such a way that the process of hydrolysis ends before it is less than complete, i.e., before the antigens are digested to free amino acids essentially free of peptides. Thus, the virus antigens still possess the required antigenic property.

Under preferred partially hydrolysis reaction conditions the instant composition is not reduced to a composition consisting of free amino acids or fragments thereof. Preferably, there is a sufficient amount of larger residues possessing immunogenic properties, these can be proteins, polypeptides and peptides. Depending on the desired clinical effect to be achieved the concentration is adjusted as necessary and without undue experimentation. The proportion of partially hydrolyzed antigen such as a protein is at least 10% by weight of the non-hydrolyzed native antigen, preferably at least 20%, preferably at least 30%, preferably at least 40%, preferably at least 50%, preferably at least 60%, preferably at least 70%, preferably at least 80%, and can be at least 90%. The meaning of 100% hydrolyzation is a situation when all proteins are hydrolyzed to free amino acids without any larger residue such as a peptide remaining after the hydrolysis reaction. Other less hydrolyzed states are “partially hydrolyzed”.

The acids that are useful for partially hydrolysis can be inorganic acids, such as hydrochloric acid, hydrobromic acid, sulfuric acid, nitric acid, carbonic acid, phosphoric acid and the like. Examples of organic acids are for example acetic acid, citric acid, propionic acid, glycolic acid, pyruvic acid, oxalic acid, malic acid, malonic acid, succinic acid, maleic acid, fumaric acid, tartaric acid, benzoic acid, cinnamic acid, mandelic acid, methanesulfonic acid, ethanesulfonic acid, salicylic acid, p-toluenesulfonic acid, and the like.

Another hydrolysis method is by providing alkaline treatment with organic or inorganic alkali solutions. The pH range in this eventuality is preferably above 8.5, more preferably between 9.5 and 15.0, more preferably between 10 and 14, and more preferably between 11 and 13.5. Additionally, the product from partial acid hydrolysis can be supplemented with alkali in order to increase the pH of the lyzate by adding an alkaline agent such as sodium hydroxide, magnesium hydroxide, calcium hydroxide, potassium hydroxide, sodium carbonate, potassium carbonate, etc. It is to be understood that the term “hydrolysis” as used herein does not mean an exposure to water, hypotonic solution, air, heat, freezing, or any gas, which may result in lysis of cells such as found in the blood.

The source of antigen does not necessarily need to be from blood or blood derivatives such as plasma or serum. One can easily adapt this invention by using sputum or other body fluids like urine, gastrointestinal secretions, liquid of tissues or tumors, synovial fluid, saliva, sputum, cyst fluid, amniotic fluid, cerebrospinal fluid, peritoneal fluid, lung lavage fluid, semen, lymphatic fluid, tears, perspirations, and prostatic fluid. These fluids can contain leukocytes, lymphocytes or other cells that are equally advantageous. Without limiting to fluids, solid tissues such as liver, pancreas, skeletal muscle, heart, kidney, lung, brain, bone marrow, tumor tissue, skin, myelin, collagen, feces, and other tissues like virus inoculated eggs, dispersed accordingly are part of preferred embodiment.

Whole blood, or serum or plasma, or cell culture medium with pathogen-infected cells present in them or recombinantly obtained hepatitis virus containing antigens are treated preferably with an acid or alkali. One skilled in the art may use other hydrolyzing agents like enzymes or detergents. Samples to be partially hydrolyzed can be pooled or from one individual, or a separate batch. Under these conditions, acid reacts with proteins or alloantigens present in the blood which also contains hepatitis viruses. Under these conditions these proteins are partially hydrolyzed and reduced to smaller fragments, which by definition are partially hydrolyzed proteins. These fragments can be polypeptides, peptides, and some amino acids. The ratio between these components is determined experimentally without excessive undue experimentation.

The duration of partially hydrolysis can be anywhere between 5 minutes to several hours, more preferably they are between 10 minutes and 5 hours, even more preferably between 20 minutes and 3 hours. The duration can be a shorter period of hours, which is determined by simple experimentation under routine hydrolysis conditions well familiar to those skilled in the art. Thus, the duration of partially hydrolysis can be 30 minutes, one hour, 1.5 hours, 2 hours, 2.5 hours, or 3 hours, or intermediate times.

In one embodiment when the composition is still in liquid solution and in process of partially hydrolysis, the products of partially hydrolysis can be precipitated with a salt of a metal or with an alkaline metal hydroxide solution that forms a salt. This step effectively ends the hydrolysis since antigens are entrapped or embedded within the matrix of the solid matter and do not react with the hydrolytic agent. The precipitation can be run simultaneously with hydrolysis or started after hydrolysis step, depending on the intended use of the final product. For example, an aqueous solution consisting of an antigen and alloantigen, preferably about 90% to about 20% protein, and more preferably about 80% to about 40% protein solution, is treated with an acid, until the pH is about 1 to about 6, preferably about 1 to about 4, more preferably about 1 to about 2. The solution is stirred at low speed, preferably at about 10 to about 500 rpm, more preferably about 30 to about 300, even more preferably about 60 to about 200 rpm for about 60 minutes to about 120 minutes, preferably about 45 to about 80 minutes, more preferably about 30 to about 60 minutes. A metal salt is then added to the stirring solution to create a slurry that solidifies as a solid mass. The mass is washed for about 1 minute to about 60 minutes, preferably about 5 to about 45 minutes, more preferably about 10 to about 30 minutes and then dried in an oven by heating at about 56 to about 200° C., preferably at about 70 to about 150° C., more preferably about 80 to about 140° C., more preferably about 100 to about 120° C., for about 1 hour to about 72 hours, preferably about 3 to about 48 hours, more preferably about 5 to about 12 hours. The dried solid mass is then reduced to a powder.

It is equally advantageous to start the precipitation and add hydrolyzant after that step. In yet another embodiment the metal salt is present prior to the partial hydrolysis and proteins are partially hydrolyzed in the presence of said metal salts, to be precipitated simultaneously and concomitantly. Representative steps for process of embedding an active ingredient such as an antigen together with alloantigen, or antigen alone without alloantigen, or alloantigen alone without the antigen, can include contacting the alloantigen with a metal salt solution to create a slurry and partially hydrolyzing the resulting mixture by contacting it with an acid so that this mixture is hardened and transforms into a solid phase or a solid state form—a process also know as a setting of the solid matter. This solid form is then reduced to powder particles with embedded within active ingredient. The sequence of these steps are easily determined before hand and can be carried out without undue experimentation as long as the final product with active ingredient is clinically effective as determined by clinical studies.

The partially hydrolyzed hepatitis antigens and alloantigens can be embedded in salts, including but not limited to: Lithium, Beryllium, Sodium, Silicone, Magnesium, Aluminium, Aluminum, Potassium, Calcium, Titanium, Vanadium, Chromium, Manganese, Cobalt, Nickel, Copper, Zinc, Arsenic, Zirconium, Molybdenum, Silver, Cadmium, Antimony, Barium, Osmium, Platinum, Gold, Mercury, Thallium, Lead, or Uranium.

Suitable soluble salts include sodium chloride, potassium chloride, calcium chloride or magnesium chloride. An example of salt of a representative metal, such as for example magnesium, is Aluminium magnesium boride, Calcium magnesium acetate, Dimagnesium phosphate, Magaldrate, Magnesium aluminide, Magnesium aspartate, Magnesium benzoate, Magnesium bromide, Magnesium carbonte, Magnesium chloride, Magnesium chloride hexahydrate, Magnesium citrate, Magnesium diboride, Magnesium diglutamate, Magnesium diuranate, Magnesium fluoride, Magnesium gluconate, Magnesium hexahydrate, Magnesium hydride, Magnesium hydroxide, Magnesium iodide, Magnesium lactate, Magnesium levulinate, Magnesium nitrate, Magnesium nitride, Magnesium orotate, Magnesium oxide, Magnesium oxychloride, Magnesium oxysulfate, Magnesium perchlorate, Magnesium peroxide, Magnesium phosphate, Magnesium pidolate, Magnesium silicide, Magnesium stearate, Magnesium sulfate, Magnesium sulfide, Magnesium sulfite, Magnesium trisilicate, Monomagnesium phosphate, Trimagnesium citrate and the like.

One skilled in the art can equally deploy salts resulting from reaction with other acids including but not limited to tosylate, methanesulfonate, acetate, citrate, malonate, tartarate, succinate, benzoate, ascorbate, alpha-ketoglutarate, alpha-glycerophosphate, formate, fumarate, propionate, glycolate, lactate, pyruvate, oxalate, oxychloride, maleate, salicyate, sulfate, sulfonate, nitrate, hydrobromate, hydrobromide, hydroiodide, and the like.

Generally, a salt of a metal can be: boride, acetate, phosphate, aluminide, aspartate, benzoate, bromide, carbonate, chloride, chloride hexahydrate, citrate, diboride, diglutamate, diuranate, fluoride, gluconate, hexahydrate, hydride, hydroxide, oxide, iodide, lactate, levulinate, nitrate, nitride, orotate, oxychloride, oxysul fate, perchlorate, peroxide, pidolate, silicide, stearate, sulfate, sulfide, sulfite, trisilicate, and the like.

In a further aspect of this invention two or more metals can be used simultaneously. For example, Calcium chloride and Magnesium chloride can be mixed at various acceptable ratios to which partially hydrolyzed antigens are embedded. Without limiting to dual metal sources it is clear that most sources contain small admixtures of plural metals or alloys thereof. For example magnesia can contain small quantities of calcium and other metals. Calcium in this mixture such as calcium carbonate does not interfere with the setting of the solid matter since higher proportion of reactive magnesia plays the predominant role. Reactive magnesium can be conveniently combined with other admixtures to create solid matters such as calcium aluminate, or other ingredients such as found for example in gypsum or cement-like compositions and that can be of significant advantage. In another embodiment naturally occurring minerals such as clay, kaolinite; chalk; silica; gypsum; feldspar; albite; anorthite; orthoclase; apatite; halite; calcite; dolomite; sodium carbonate; siderite; biotite; bentonite; muscovite; chlorite; stilbite; pyrite; hematite and alike that can be dispersed and made into a slurry into which antigen is admixed and partially hydrolyzed.

In case partial hydrolysis happens in alternative step such as after or simultaneously with the embedding step of the antigen, this then will classify as the accelerant-like reaction that can be helpful in the composition of the present invention. Examples of chemicals that are useful, can be for example alkaline chemicals which mobilize the antigen within the matrix, acids and salts of acids as described above, and proteins, peptides and amino acids serving as organic accelerators of the reaction. Alkaline chemicals that are part of the invention include chemicals such as alkali and alkali earth hydroxides, carbonates, formates, aluminates and silicates. The dosages of such alkaline accelerants are determined without undue experimentation and are part of the composition of the present invention. The most effective for example can be sodium silicate and sodium aluminate as convenient reagents.

The preferred hydrolyzants are acids and soluble salts of acids and generally both the cation and anion contribute to the overall effect. Some anions can cause significant advantage such as halides, chlorides, nitrates, nitrites formates, thiosulfates and thiocyanates. It did not escape the attention that divalent and trivalent cations such as calcium, magnesium, barium and aluminium appear to be far more attractive than monovalent ions such as sodium, potassium and ammonium. Other acidic reactants include ferrous sulphate and calcium or aluminium sulphate, calcium chloride and the like.

These methods are equivalent in recovery and creation of the solid matter. The solid matter can be further processed, e.g., heat treated, made into a powder, and compounded into granules, pills, or tablets. These steps can be in any logically acceptable order. The reduced or partially hydrolyzed form is inherently the part of the body of antigen.

According to one of the embodiments the partially hydrolyzed antigen keeps its physical structure and shape such as their molecular and supramolecular shape close to the initial three-dimensional form or conformational integrity. According to another embodiment the reduced form of antigen is at variation with the initial structure but can nevertheless display the same antigenicity. The difference between present composition and prior art compositions having an antigen mainly adsorbed to the surface of an immune adjuvant such as aluminum hydroxide is that the instant antigen is securely embedded within the matrix, which allows it to keep the physical structure intact even at conditions that are commonly unfavorable, i.e., high temperature at longer periods of time.

It is well known that exposure to high temperature, for example, heating at 56° C. or higher, like 80° C. for one hour or more, can inactivate HBV, HCV, HIV, HAV and other viruses. Such a thermal treatment or denaturation effectively destroys the infectivity and at the same time annuls the antigenicity, since antigens of a virus (e.g., proteins, sugars, lipids, glycoproteins, amino and nucleic acids) are rendered irreversibly inactivated by heat out of their original native structure. These misshapen antigens are considered by those skilled in the art as useless since using them in a vaccine preparation will be a futile exercise. The immune system trained by such misshapen antigens will be not able to recognize native antigens such as found on live virus in a patient and therefore the immune response will be misdirected. This has been and remains the cornerstone of vaccinology. Thus, exposure to temperatures above 80° C. termed as “high temperatures” and for prolonged periods of time is useful to destroy the infectious virus but not useful for making vaccines. However, surprisingly, the instant composition, when exposed to high temperatures has not lost its antigenicity and shows positive clinical benefit. Thus, instant composition can be safely used since potentially infectious virus that can be present within the composition is effectively killed by exposure to high temperatures for one hour or more. The advantage of thermal treatment, while obvious from the standpoint of elimination of infectious viruses, is not at all obvious from the standpoint of conservation of antigenicity—an observation that is contrary to the consensus prevailing in the art.

This advantage also eliminates the need for so-called “cold chain” requirements that are characteristic to classical vaccines that need to be refrigerated. The instant composition is stable for years and can withstand storage at ambient, such as room or tropical temperatures, without any deleterious effect on its biological activity. In particular, the composition is stable for at least one year at room temperature, more preferably for at least 1.5 years at room temperature, and even more preferably more than 2 years at room temperature.

The assembly of these antigens in powder or small granule microparticles and/or nanoparticles effectively provides the source for further transformation such as combining with tablet excipients. An excipient is generally viewed as an inert substance which is added to an active ingredient to provide bulk, for example in tablets. These can be any of the following ingredients, or compounds of a similar nature: a binder such as microcrystalline cellulose, gum or gelatin; an excipient such as starch or lactose, a disintegrating agent such as alginic acid or corn starch; a lubricant such as magnesium stearate; a glidant such as colloidal silicon dioxide; a sweetening agent such as sucrose or saccharin; or a flavoring agent such as peppermint or some other flavoring. Mixing with excipients is done by wet or dry granulation processes well known to formulation experts and resulting mixture is compressed into tablets. Various properties of excipients, such as moisture content, particle size and distribution, polymorphism, amorphism, crystal habit, hydration state, and lubricant and binder level of the blend that have an influence on compaction are within routine knowledge of a practitioner. Tableting speed and mechanistic aspects of tableting such as choice of punches/dies, tableting machines can be selected without undue experimentation by artisans. In addition, dosages can contain various other materials which modify the physical form, for example, coatings of sugar, shellac, or other enteric agents.

The tablets can be covered with enteric coating material that is predominantly soluble in the intestinal fluid, but substantially insoluble in the gastric fluids of stomach. Examples include polyvinyl acetate phthalate (PVAP), hydroxypropylmethylcellulose acetate succinate (HPMCAS), cellulose acetate phthalate (CAP), methacrylic acid copolymer, hydroxypropylmethylcellulose succinate, cellulose acetate succinate, cellulose acetate hexahydrophthalate, hydroxypropylmethylcellulose hexahydrophthalate, hydroxypropylmethylcellulose phthalate (HPMCP), cellulose propionate phthalate, cellulose acetate maleate, cellulose acetate trimellitate, cellulose acetate butyrate, cellulose acetate propionate, methacrylic acid/methacrylate polymer, methacrylic acid-methyl methacrylate copolymer, ethyl methacrylate-methylmethacrylate-chlorotrimethylammonium ethyl methacrylate copolymer, and the like, and combinations comprising one or more of the foregoing enteric polymers. Other examples include natural resins, such as shellac, SANDARAC, copal collophorium, and combinations comprising one or more of the foregoing polymers. Yet other examples of enteric polymers include synthetic resin bearing carboxyl groups, e.g., the methacrylic acid:acrylic acid ethyl ester copolymers. Preferably enteric coating material is chosen from those that are commercially available, although new materials can be selected that are with the skill of practicing artisan.

The preferred dosage form is an oral solid dosage form such as a tablet or pill. Moreover, the pharmaceutical composition described herein can be formulated into any suitable dosage form, including but not limited to, aqueous oral dispersions, aqueous oral suspensions, aerosols, controlled release formulations, fast melt formulations, effervescent formulations, self-emulsifying dispersions, solid solutions, liposomal dispersions, lyophilized formulations, capsules, powders, delayed release formulations, immediate release formulations, modified release formulations, extended release formulations, pulsatile release formulations, multiparticulate formulations, and mixed immediate release and controlled release formulations.

Equipment used for these processes is commercially available. One skilled in the art can easily select appropriate ones among Rotary Drums, Sectional Coolers, (Drying and Cooling) Flash Dryers, Fluidized Bed Plants, Turbo-Tray Dryers, Solidizer, Disc Dryers, Spirocorn, Belt Dryers, Tray Dryers Simplicior and Favorit, Air Mix, Spray Mix, Mills and Classifiers and order them from various manufacturers like Duske Engineering, Inc. Fenwal, Baxter, among many others.

In other embodiments of the present invention, the amount of active ingredient in the tablet, i.e., partially hydrolyzed viral antigens with alloantigen, administered to a subject via a solid dosage form, such as a tablet or pill, is the amount known in the art to achieve a therapeutically effective concentration of drug of a human or animal in need thereof. For example, the amount of one or more active ingredients may range from about 0.001 micrograms to about 1 g. In other embodiments, the amount of active ingredients may range from about 0.01 micrograms to about 100 mg. In other embodiments, the amount of active ingredients may preferably range from about 0.1 microgram to about 10,000 microgram, from about 0.5 microgram to about 1,000 microgram or from about 1 microgram to about 500 microgram. In another embodiment, a formulation is administered in a solid dosage form at a concentration of about 10 microgram to about 500 microgram. In another embodiment, the active ingredients formulation is administered in a solid dosage form at concentration of about 1-100 microgram. In other embodiment the amount of active ingredients is from about 0.00001% to about 30% based on the total weight of the composition, preferably the amount is between about 0.001% and 20%, preferably the amount is between about 0.01% and 10%, preferably the amount is between about 0.1% and 5%. The content can also vary depending on the intended use. For therapeutic use the content can be higher by about one order of magnitude from content of the composition intended for prophylactic use. For example, if in therapeutic composition the quantity of HBV surface antigen (HBsAg) equivalent is 60 microgram then in prophylactic version the dose can be reduced to 5 or 10 micrograms. However, this is not an absolute requirement. These and other implications related to effective dose are not intended to be limiting and are provided by a way of example only. The precise amount of active ingredients that is “therapeutically effective” is determined by standard clinical testing whereby the clinical effect is monitored by means well known to those skilled in the art.

In some embodiments, formulations provide a therapeutically effective amount of composition over an interval of about 3 hours to about 24 hours after administration, enabling, for example, once-a-day, twice-a-day (b.i.d.), or three times a day (t.i.d.) administration if desired. Simple dosing regimen of one or two tablet per day is preferred. Administration can be single tablet administration, more preferably repeated administration for at least one week up to one month to one year, or carried out for years as deemed necessary. Treatment can be stopped after 1-3 months and repeated again after one month of break, although in some patients repeat treatment is not necessary for as long as 3 or 6 months intervals. Some would prefer to stop for one year and then repeat again. In some individuals treatment may not to be repeated at all as patients are cured and need no further dosing.

In another aspect of the invention the composition is administered without an immune adjuvant. While this is the preferred mode of administration, one skilled in the art, can administer the composition with an immune adjuvant known in the art. Preferred adjuvants are those that aid in creating the state of immune tolerance. Toll-like receptor (TLR) adjuvants such as zymosan and vitamin A are representative examples of such adjuvants. One skilled in the art will be able to find equivalent adjuvants. Examples of other immune adjuvants include but not limited to bacterial adjuvants; Cytokine adjuvants; plant-derived adjuvants and alike. Examples of synthetic adjuvants are Threonyl muramyl dipeptide; adamantylamide dipeptide (AdDP); Capric acid; polycationic sphingolipid, N-palmitoyl D-erythro-sphingosyl carbamoyl-spermine (ceramide carbamoyl-spermine, CCS); 1,25-Dihydroxyvitamin D3; and Avridine—synthetic lipoidal amine. Examples of bacterial adjuvants are E. coli thermolabile toxin (LT) and its derivatives LTK63, LTR72 and R192G; cholera toxin (CT); B-subunit of CT (CTB); Brucella lumazine synthase; Shigella invasin complex; Bacillus anthracis edema toxin; Vibrio vulnificus flagellin FlaB; outer membrane protein of Neisseria meningitides; Protollin (Neisseria meningitidis outer membrane proteins non-covalently complexed with Shigella flexneri 2a lipopolysaccharide). Examples of TLR ligands, which are type I transmembrane proteins that recognize microorganisms which come in contact with intestinal tract mucosa, are TLR-2/6 agonist MALP-2; Double-stranded polyriboinosinic-polyribocytidylic acid (poly(I:C); CpG-containing oligodeoxynucleotide (CpG ODN); and TLR-4 agonists such as aminoalkyl glucosaminide phosphates (AGPs). Examples of Cytokine adjuvants are Type I interferons (IFN); IL-12; Flt3 ligand (Flt3L); IL-1alpha; granulocyte macrophage colony-stimulating factor (GMCSF); cytotoxic T lymphocyte antigen-4 (CTLA-4); macrophage inflammatory protein-1alpha (MIP-1alpha); indoleamine 2,3-dioxygenase; CD40L and other cytokines which are reported as potent adjuvants. Examples of plant-derived adjuvants are Phaseolus vulgaris phytohaemagglutinin (PHA); mistletoe lectins (MLI, MLII, MLIII); tomato lectin (LEA); wheat germ agglutinin (WGA); Ulex europaeus lectin 1 (UEA-1); detergent-like substance saponin QS-21, from the bark of the South American soap tree; another Quillaja saponaria derivative, Quil A; quillaja saponin derivative GPI-0100; Gypsophila sp. Saponin; Chenopodium quinoa; Polygala tenuifolia; apiogalacturonanic pectin of duckweed (Lemna minor), Lemnan LM; Solanum torvum (STE); Ricin B; birch (Betula alba) pollen; Thujae summitates, Baptisiae tinctoriae radix, Echinaceae purpureae radix and Echinaceae pallidae radix; Ginkgo biloba; and Momordica charantia (bitter gourd). Examples of Epitope adjuvants are for example amino acid residues 830-843 of the tetanus toxoid (TTE), 1-20 of tuberculosis bacteria heat shock protein 65 (TBE), 54-65 of rubella protein E2-4 (ME) and 35-48 of trachoma heat shock protein 60 (CE); MSP I (DYDVVYLKPLAGMYK) of P. vivax; and DR-restricted helper T-cell epitope known as PADRE. Other adjuvants well known in the art are alum, aluminum, mineral oils, metabolizable organic oils, MF59, ISCOMs, Adjuvant 65 (containing peanut oil, mannide monooleate and aluminum monostearate), the pluronic polyol L-121, emulsifiers such as Freund's incomplete adjuvant and squalene (Shark-oil). It is understood that when metal-based immunological adjuvants such as aluminum salt or alum are used, they are not part of the metal matrix utilized for embedding the antigen.

Upon digestion in the gut the matrix releases the original parts or fragments of this antigen and presents it to the immune system of a host. The host processes the antigen via cellular pathways, e.g., antigen presentation via MHC Class 1 or 2 by specialized mucosal and/or dendritic cells functioning as antigen presenting cells (APC). While the mechanism of the invention relating as to how the claimed composition might work is not the required part of the invention, this provides a substance that can be of interest to those skilled in the art, which by no means has no effect on the thrust and gist of the invention which is supported by clinical evidence in foregoing examples.

In a preferred embodiment, partially hydrolyzed hepatitis virus antigens are present in the composition together with alloantigen(s), which will be useful for prophylactic and therapeutic applications for preventing or actively treating hepatitis and hepatitis virus infection. In prior art of vaccines considerable effort is made to purify antigens from non-viral antigens. This is due to concern that when injected these non-viral antigens cause undesirable immune reactions that can be sometimes life-threatening. Thus calling for alloantigens to be present in the composition together with virus antigens is against common wisdom prevailing in the art. Alloantigen can be for example albumin, MHC molecules, cytokine, lymphokine, chemokine, immunoglobulin and other molecules present in a host either one-by-one or preferably in a combination. The ratio between partially hydrolyzed hepatitis virus antigens and alloantigen(s) is determined by art known methods without undue experimentation. Preferably, the ideal ratio is one that is found naturally, as for example in the whole blood, i.e., the ratio between a quantity of hepatitis viruses and other host molecules such as the amount of total proteins found in the blood. The same applies to a situation in which virus or its antigen is recombinantly expressed in propagating cells, like yeast cells or mammalian cells such as CHO cells. These recombinants are well known, for example, a third generation HBV vaccine containing pre-S1, pre-S2, and S antigenic components of both viral surface antigen are produced in a continuous mammalian cell line, the mouse c127 clonal cell line, after transfection with recombinant hepatitis B surface antigen DNA. In situations when composition components are grown in cells of species other than human but are intended to be used in humans the alloantigen is then termed xenoantigen, which purposes of this invention can be called alloantigen. In other situations, when it is required, one can easily grow not the part of a virus, but the entire virus, for example HEV virus grown in human lung carcinoma A549 cell culture. Notwithstanding the above reasoning about preferred ratio one can deliberately increase the amount of alloantigen or antigen. In any case the amount of alloantigen shall not be too low so that it could be considered as a contaminant such found for example in standard vaccines, such as hepatitis B vaccine made from plasma. While the amount of contaminating plasma proteins, i.e., alloantigens, in hepatitis B vaccine made from pooled plasma varies depending on a batch or manufacturer it is usually less than 1%. In the case of hepatitis A vaccine the WHO recommends the amount of contaminating albumin to be less than 50 nanograms per dose (see by way of example the link at the WHO site http://www.who.int/biologicals/publications/trs/areas/vaccines/hepatitis/WHO_TRS_{—}858_A 2.pdf). Thus, these requirements distinguish the instant composition from classical vaccine compositions of prior art, since in the present invention the ratio of total alloantigen to antigen is always higher than 1:100 and always exceeds 50 nanogram level when albumin is used to gauge the amount of allogeneic material. Such compositions, especially in oral form of delivery, will be useful to a host in need thereof, such as a human host or other species in need thereof. This invention can encompass hepatitis viruses as well as viruses that are related to them. For example hepatitis C related flaviviruses (Flaviviridae) include, without limitation: Absettarov; Alfuy; Apoi; Aroa; Bagaza; Banzi; Bououi; Bussuquara; Cacipacore; Carey Island; Dakar bat; Dengue viruses 1, 2, 3 and 4; Edge Hill; Entebbe bat; Gadgets Gully; Hanzalova; Hypr; Ilheus; Israel turkey meningoencephalitis; Japanese encephalitis; Jugra; Jutiapa; Kadam; Karshi; Kedougou; Kokoera; Koutango; Kumlinge; Kunjin; Kyasanur Forest disease; Langat; Louping ill; Meaban; Modoc; Montana myotis leukoencephalitis; Murray valley encephalitis; Naranjal; Negishi; Ntaya; Omsk hemorrhagic fever; Phnom-Penh bat; Powassan; Rio Bravo; Rocio; Royal Farm; Russian spring-summer encephalitis; Saboya; St. Louis encephalitis; Sal Vieja; San Perlita; Saumarez Reef; Sepik; Sokuluk; Spondweni; Stratford; Temusu; Tyulenly; Uganda S, Usutu, Wesselsbron; West Nile; Yaounde; Yellow fever; and Zika.

Other human viruses that can be found circulating in the blood are equally suitable to be used in the instant composition either alone or in combination with hepatitis viruses of interest which belong to Hepadnaviridae (Hepatitis B virus); Picornaviridae (e.g. hepatitis A virus; and related polio viruses, enteroviruses, human Coxsackie viruses, rhinoviruses, echoviruses); Flaviridae (e.g. dengue viruses, encephalitis viruses, yellow fever viruses). Examples of viruses include but are not limited to: Retroviridae (e.g. human immunodeficiency virus); Caliciviridae (e.g. strains that cause gastroenteritis); Togaviridae (e.g. equine encephalitis viruses, rubella viruses); Coronoviridae (e.g. coronaviruses); Rhabdoviradae (e.g. vesicular stomatitis viruses, rabies viruses); Filoviridae (e.g. ebola viruses); Parantyxoviridae; Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arenaviridae (hemorrhagic fever viruses); Reoviridae (e.g. reoviruses, orbiviurses and rotaviruses); Birnaviridae; Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxyiridae (variola viruses, vaccinia viruses, pox viruses); and Iridoviridae (e.g. African swine fever virus); and unclassified viruses (e.g. the etiological agents of Spongiform encephalopathies.

The instant preparation is not directed exclusively against viruses. Antigens of any pathogen that is found in a host can be useful to induce adequate immune response. For example, contemplated to treat fungal infection, then fungal antigens may be derived from Dermatophytres, including: Epidermophyton floccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T. verrucosum var. album, var. discoides, var. ochraceum, Trichophyton violaceum, and/or Trichophyton faviforme. Without limiting to these fungi other fungal pathogens of interest include Aspergillus fumigatus, Aspergillus flavus, Aspergillus niger, Aspergillus nidulans, Aspergillus terreus, Aspergillus sydowi, Aspergillus flavatus, Aspergillus glaucus, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomnyces dermatidis, Cryptococcus neoformans, Geotrichum clavatum, Histoplasma capsulatum, Pneumocystis carinii, Klebsiella pneumoniae, Paracoccidioides brasiliensis, Pityrosporum ovale, Pythiumn insidiosum, Sacharomnyces cerevisae, Saccharomyces boulardii, Saccharomyces pombe, Scedosporium apiosperum, Sporothrix schenckii, Trichosporon beigelii, Toxoplasma gondii, Penicillium marnaeffei, Malassezia spp., Fonsecaea spp., Wangiella spp., Sporothrix spp., Basidiobolus spp., Conidiobolus spp., Rhizopus spp, Mucor spp, Absidia spp, Mortierella spp, Cunninghamella spp, Saksenaea spp., Alternaria spp, Curvularia spp, Helminthosporium spp, Fusarium spp, Aspergillus spp, Penicillium spp, Monolinia spp, Rhizoctonia spp, Paecilomyces spp, Pitliomyces spp, and Cladosporium spp.

The composition is equally useful to treat other microbial infections that are found in the blood or body fluids of a host including for example Staphylococcus aureus, Staphylococcus epidermidis, and Staphylococcus sciuri, Sinorhizobium species (e.g., meliloti), Listeria species (e.g., monocytogenes), Clostridium species (e.g., acetabutylicum, difficile), Vibrio species (e.g., cholerae), Corynebacterium species (e.g., diptheriae), Brucella species (e.g., suis), Pseudomonas species (e.g., aeruginosa, syringae, putida), Shewanella species (e.g., putrefasciens), Mesorhizobium species (e.g., loti), Caulobacter species (e.g., crescentus), Lactococcus species (e.g., lactis), Mycobacterium species (e.g., smegmatis), Burkholderia species (e.g., mallei, pseudomallei), Geobacter species (e.g., sulfurreducens), Treponema species (e.g., denticola), Bacillus species (e.g., stearothermophilus, anthracis, subtilis, halodurnas), Escherichia species (e.g., coli), Enterococcus species (e.g., faecalis), Salmonella species (e.g., dublin, enteriditis, paratyphi, typhi), Klebsiella species (e.g., pneumoniae), Bordetella species (e.g., parapertussis), Actinobacillus species (e.g., actinomycetemcomitans), Streptomyces species (e.g., coelicolor), Streptococcus species (e.g., pyogenes, pneumoniae), Yersinia species (e.g., pestis), Agrobacterium species (e.g., tumefkiens) and Acinetobacter species.

This invention also encompasses treatment of autoimmune diseases regardless whether they are caused by microbial or viral infections or have unknown etiology. These for example include: Acute necrotizing hemorrhagic leukoencephalitis, Addison's disease, Agammaglobulinemia, Allergic asthma, Allergic rhinitis, Alopecia greata, Amyloidosis, Ankylosing spondylitis, Anti-GBM/Anti-TBM nephritis, Antiphospholipid syndrome (APS), Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune thrombocytopenic purpura (ATP), Autoimmune thyroid disease, Axonal & neuronal neuropathies, Balo disease, Behcet's disease, Bullous pemphigoid, Cardiomyopathy, Castleman disease, Celiac sprue (nontropical), Chagas disease, Chronic fatigue syndrome, Chronic inflammatory demyelinating polyneuropathy (CIDP), Churg-Strauss syndrome, Cicatricial pemphigoid/benign mucosal pemphigoid, Cogans syndrome, Cold agglutinin disease, Congenital heart block, Coxsackie myocarditis, CREST disease, Crohn's disease, Demyelinating neuropathies, Dermatomyositis, Devic disease, Discoid lupus, Dressler's syndrome, Endometriosis, Eosinophilic fasciitis, Erythema nodosum, Essential mixed cryoglobulinemia, Evans syndrome, Experimental allergic encephalomyelitis, Fibromyalgia, Fibrosing alveolitis, Giant cell arteritis (temporal arteritis), Goodpasture's syndrome, Graves' disease, Guillain-Barre syndrome, Hashimoto's disease, Hemolytic anemia, Henoch-Schonlein purpura, Herpes gestationis, Hypogammaglobulinemia, Idiopathic pulmonary fibrosis, Idiopathic thrombocytopenic purpura (ITP), IgA nephropathy, Immunoregulatory lipoproteins, Inclusion body myositis, Insulin-dependent diabetes (typel), Interstitial cystitis, Juvenile arthritis, Juvenile diabetes, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neutropenia, Ocular cicatricial pemphigoid, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Pars planitis (peripheral uveitis), Parsonnage-Turner syndrome, Pemphigus, Peripheral neuropathy, Perivenous encephalomyelitis, Pernicious anemia, POEMS syndrome, Polyarteritis nodosa, Polymyalgia rheumatica, Polymyositis, Postmyocardial infarction syndrome, Postpericardiotomy syndrome, Primary biliary cirrhosis, Progesterone dermatitis, Psoriasis, Psoriatic arthritis, Pure red cell aplasia, Pyoderma gangrenosum, Raynauds phenomenon, Reflex sympathetic dystrophy, Reiter's syndrome, Relapsing polychondritis, Restless legs syndrome, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Sympathetic ophthalmia, Takayasu's arteritis, Temporal arteritis/Giant cell arteritis, Thrombocytopenic purpura (TTP), Tolosa-Hunt syndrome, Transverse myelitis & necrotizing myelopathy, Type I, II, & III autoimmune polyglandular syndromes, Ulcerative colitis, Undifferentiated connective tissue disease (UCTD), Uveitis, Vasculitis, Vesiculobullous dermatosis, Vitiligo, Wegener's granulomatosis, and the like.

Without further limitation the preferred embodiments of the invention contemplate Mycoplasma infections like Mycoplasma pneumoniae, Ureaplasma urealyticum, Mycoplasma genitalium, Legionellosis, Legionella pneumophilia, Yersinia pestis, Leptospirosis, Leptospira, Rat-Bite Fever (Haverhill Fever), Streptobacillus moniliformis, Tick-Borne Diseases. Spirochetes: Borrelia burgdorferi, Erythema migrans, Acrodermatitis Atrophicans, Borrelial Lymphocytoma, Relapsing Fever, Ehrlichiosis, Tularemia. Chlamydia infections, Chlamydia pneumoniae, Ornithosis, Psittacosis, Parrot Fever, Chlamydia psittaci, Bartonella Infections, Q Fever. Rickettsia infections such as Rocky Mountain Spotted Fever, Typhus, Epidemic Louse-Borne Scrub typhus. Treponema pallidum, Vibrio Infections and alike. Intracellular pathogens such as Mycobacterium tuberculosis, Mycobacterium leprae and the like.

The instant invention can be a combined vaccine having viral and microbial antigens. These supplementary antigens can be selected from the group consisting of: diphtheria toxoid (D), tetanus toxoid (T) acellular pertussis antigens (Pa), inactivated polio virus (IPV), haemophilus influenza antigen (Hib), hepatitis A antigen, herpes simplex virus (HSV), chlamydia, GSB, HPV, streptococcus pneumoniae and neisseria antigens. Antigens conferring protection for other diseases may also be combined in the vaccine formulation of the present invention.

Another preferred embodiment of the invention is a method of treating autoimmune diseases which may or may not relate to viral infections. These include hepatitis that is caused by hepatitis viruses; autoimmune hepatitis; parvovirus B 19 infection-associated anemia; autoimmune hemolytic anemia; allogeneic bone marrow or organ transplant recipients; diabetes; fetal alloimmune thrombocytopenia; pediatric intractable epilepsy; hypogammaglobulinemia; X-linked agammaglobulinemia; common variable immunodeficiency; Wiskott-Aldrich syndrome; idiopathic thrombocytopenia purpura; Kawasaki syndrome; dermatomyositis; Alzheimer disease, multiple sclerosis; multiple myeloma; myasthenia gravis; polymyositis; Guillain-Barré syndrome; inflammatory demyelinating polyneuropathy; lupus erythematosus; Lambert-Eaton myasthenia; pemphigus foliaceus, and alike. One skilled in the art will easily recognize and adopt the instant invention to treat other autoimmune diseases such as Acute disseminated encephalomyelitis; Addison's disease; Alopecia areata; Ankylosing spondylitis; Antiphospholipid antibody syndrome; Autoimmune hemolytic anemia; Autoimmune hepatitis; Anti-Smooth Muscle Actin; Autoimmune inner ear disease; Bullous pemphigoid II Anti-Bullous Pemphigoid Antigen 1 and 2 (Hemidesmosome antigens); Coeliac disease; Chagas disease; Chronic obstructive pulmonary disease; Crohns Disease; Dermatomyositis; Diabetes mellitus; Endometriosis; Goodpasture's syndrome; Anti-Basment Membrane Collagen Protein; Heart ischemia and stroke; Graves' disease; Guillain-Barré syndrome; Anti-ganglioside; Hashimoto's disease; Hidradenitis suppurativa; Kawasaki disease; IgA nephropathy; Idiopathic hrombocytopenic purpura; Interstitial cystitis; Lupus erythematosus; Mixed Connective Tissue Disease; Morphea; Multiple sclerosis; Anti-Myelin Basic Protein; Myasthenia gravis; Narcolepsy; Neuromyotonia; Pemphigus Vulgaris; Anti-Desmogein; Periodontitis; Pernicious anaemia; Polymyositis; Primary biliary cirrhosis; Anti-p62, Anti-sp100, Anti-Mitochondrial; Psoriasis; Rheumatoid arthritis; Rheumatoid factor; Schizophrenia; Scleroderma; Anti-topoisomerase; Sjogren's syndrome; Stiff person syndrome; Temporal arteritis; Ulcerative Colitis; Idiopathic inflammatory bowel disease; Vasculitis; Vitiligo; Wegener's granulomatosis and the like.

The composition of the present invention can be administered in combination with another anti-hepatitis virus drug. The dosages given will depend on absorption, inactivation, and excretion rates of the drug as well as other factors known to those of skill in the art. Examples of antiviral drugs are described in the Background section of this invention as well as in patent literature such as for example in U.S. Pat. No. 7,456,155 incorporated herein by way of reference. It is also not without consequence that did not escape the attention is the possibility of using instant invention in combination with any other bioactive substances such as pharmaceutically effective substances, including, but not limited to, antiinflammatory drugs, analgesics, tranquillizers, antianxiety drugs, antispasmodics, antidepressants, antipsychotics, antianxiety drugs, narcotic antagonists, antiparkinsonism agents, cholinergic agonists, chemotherapeutic drugs, immunosuppressive agents, antiviral drugs, antimicrobial drugs like antibiotics, appetite suppressants, anticholinergics, antimetrics, antihistaminics, antimigraine agents, coronary, cerebal or peropheral vasodilators, hormonal agents, contraceptives, antithrombotic agents, diuretics, antihypertensive agents, cardiovascular drugs, opioids, and the like.

Without limiting to drugs, other substances can be equally used in combination with instant composition such as subcellular compositions like nucleus or mitochondria, cells, plasmids, bacteria, viruses, outer membrane vesicle preparations, and molecules including but not limited to, lipids, organics, proteins and peptides (synthetic and natural), peptide mimetics, natural hormones (like hCG, FSH, LH, peptide hormones, and steroids), D and L amino acid polymers, oligosaccharides, polysaccharides, nucleotides, oligonucleotides and nucleic acids, including DNA and RNA, protein nucleic acid hybrids, small molecules and physiologically active analogues thereof. Further, the modifiers may be derived from natural sources or made by recombinant or synthetic means and include analogues, agonists and homologues.

Other features and advantages of the present invention are apparent from the additional detailed disclosure as provided below and which includes different examples. The examples provided below illustrate different components and methodology useful in practicing the present invention. The examples do not limit the claimed invention. Based on the present disclosure one skilled in the art can identify and employ other components and methodology to practice the present invention, which however will be obvious from the disclosure of the present invention.

### EXAMPLES

### Example 1

### Acute Toxicity in Animals

Laboratory mice are force-fed with excess doses of instant composition for 2 weeks as per standard test for acute toxicity to identify 50% lethal dose (LD50). However, no deaths are observed in mice population at maximum tested daily dose of preparation equivalent to about 27 g/kg. If this dose is extrapolated to an average size human it corresponds to 1.9 kg of product per day—a dose equivalent to 2,000 tablets per day. Furthermore, no discernible morphological changes in organs are observed by autopsy. Thus, the LD50 could not be established.

### Example 2

### Chronic Toxicity in Animals

No treatment-related deaths are observed in female and male rats during the course of a chronic toxicity study with the composition of Example 1, even at the highest tested 2.5% w/w dose. The increase in water consumption accompanied by soft stool is observed among some animals in 2.5% group. This seemed to produce slight but not statistically significant reduction in normal body weight gain but only among high-dosed males. Overall there are no perceptible changes in eating habits, behavior, symptoms, morphology, and weight of organs in any dose treated-group.

### Example 3

### Safety Study in Humans

The instant oral dosage composition having partially hydrolyzed HBV and HCV antigens from chronic hepatitis carriers is given to normal human volunteers for duration of 2 months at daily dose of 1-2 tablets. The blood biochemistry and other standard measures of liver and kidney function, as well as immune system parameters are taken before and after dosing is finished. The results are analyzed statistically and presented in Table 1. As can been seen from results the instant composition is safe, without any deleterious effect on any parameters. In some individuals CD4 T lymphocyte counts are found rising indicating positive effect on cellular immunity. Of interest is the observation that there is an improvement in liver function of those individuals who are suspected to abuse alcohol. In such individuals the instant composition is effective in improving indices of alcohol-induced hepatitis, especially in decompensated cirrhotic patients.

TABLE 1
Results of safety study as measured by various lab parameters

| | | | | P value |
|---|---|---|---|---|
| | | | Normal | (Student |
| Parameter | Before | After | Range | t-test) |
| BUN (mg/dL) | 13.5 | 12.7 | 7-21 | 0.23 |
| Creatinine (mg/dL) | 0.96 | 0.98 | 0.5-1.4 | 0.72 |
| Total protein (g/dL) | 7.55 | 7.61 | 6.3-8.2 | 0.71 |
| Albumin (U/L) | 4.35 | 4.31 | 3.5-4.8 | 0.6 |
| Globulin (g/dL) | 3.18 | 4.23 | 1.5-4.5 | 0.1 |
| Hemoglobin (g/dL) | 122 | 121 | 12-16.2 | 0.2 |
| Hematocrit (%) | 36 | 38 | 37-52 | 0.3 |
| Total bilirubin | 0.7 | 0.68 | 0.2-1.3 | 0.0004 |
| (mg/dL) | | | | |
| Direct bilirubin | 0.17 | 0.1 | <0.3 | 0.001 |
| (mg/dL) | | | | |
| AST (U/L) | 24.5 | 20.1 | 5-35 | 0.06 |
| ALT (U/L) | 26.6 | 25.6 | 7-56 | 0.3 |
| Alk. phosphatase | 95 | 65 | 38-126 | 0.0005 |
| (U/L) | | | | |
| Platelets (1000/μL) | 217 | 218 | 140-450 | 0.6 |
| WBC (cells/μL) | 5,592 | 5,327 | 4,100-10,900 | 0.2 |
| Neutrophils (%) | 51.7 | 50.9 | 40-70 | 0.8 |
| Eosinophils (%) | 5.6 | 5.4 | 0-7 | 0.8 |
| Monocytes (%) | 4.3 | 5.9 | 2-12 | 0.03 |
| Basophiles (%) | 1.3 | 0.9 | 0-2 | 0.06 |

### Example 4

### Prophylactic Use

Propagated in vitro avian liver cells, LMH, expressing the duck hepatitis B virus (DHBV) core antigen (DHBcAg) are partially hydrolyzed in their entirety, embedded into metal salt matrix and made into solid form pellets admixed with bird feed, which is then fed to ducks. After one month the ducks are challenged by intravenous inoculation with infectious DHBV. Liver biopsies obtained on day 4 post-challenge demonstrate that vaccination did not prevent infection of the liver as similar proportions of infected hepatocytes are detected in all vaccinated and non-vaccinated control ducks. However, surprisingly, the analysis of liver tissue obtained at 10 or more days post-challenge reveals that 10 out of 13 of vaccinated ducks cleared the DHBV infection without any detectable infected cells or virus. In contrast, 11 out of 13 unvaccinated ducks in control group had shown chronic DHBV infection. The difference in outcome between control and vaccinated groups is highly significant as tested by Fisher's 2×2 exact test with P=0.0048. Thus, vaccination of ducks with partially hydrolyzed cells having recombinant DHBcAg elicits adequate immune responses resulting in rapid resolution of DHBV infection. Same results are obtained when yeast expressing human HBV antigen spiked with HBsAg expressed in plant cell culture and duck liver cell culture is fed to birds instead of DHBV.

### Example 5

### Immunotherapy of Hepatitis

Twenty patients with chronic hepatitis C diagnosis are selected and are given daily one-to-two tablets of the instant composition for one month. The composition is obtained from blood of hepatitis virus-infected donors and processed by steps of hydrolysis, heating, and precipitation with magnesium salt, after which it is formulated into a tablet. These patients in addition to HCV are also infected with Mycobacterium tuberculosis (TB) and HIV and treated with TB drugs concomitantly. After one month physical, clinical and lab analysis results are obtained and compared with entry level (“before”) parameters. As shown in Table 2 all patients are improved considerably, except one patient. These results demonstrate that besides prophylactic effect, the instant composition is highly effective as immunotherapy. These are evidenced by disappearance of clinical symptoms of hepatitis such as abdominal pain, fatigue and jaundice. Specific hepatitis signs such as enlarged liver, elevated transaminase (ALT) and bilirubin are all back to normal levels after dosing. It is of interest that after one month TB and HIV conditions are also improved, indicating that immunotherapy is not limited to hepatitis only and is effective in treating other viral and bacterial infections. This is particularly evident considering that after one month the majority of control patients who are treated exclusively with TB or AIDS drugs failed to improve noticeably. Of particular noted benefit of the composition are disappearance of Mycobacterium tuberculosis from sputum samples within one month in 19 out of 20 patients, increased levels of hemoglobin back to normal levels, and impressive body weight gain. Also preparation shows potent anti-inflammatory and anti-pyretic activities as evidenced by reduced erythrocyte sedimentation rate, reduced abnormal leukocyte counts, and rapid reversion of fever back to normal body temperature within 1-3 days after dosing. Interestingly, following treatment patients became free of athletes foot fungal infection.

TABLE 2
Baseline and outcome characteristics of hepatitis patients
with TB and HIV co-infections treated for one month

| | | | TB | Liver size | | Erythrocyte | | | |
|---|---|---|---|---|---|---|---|---|---|
| | | | type/ | in cm over | | sedimentation | | Leukocyte × | |
| Patient | | | AIDS | normal | | rate (ESR) | | 10⁹ L | |
| No. | Sex | Age | stage | Before | After | before | After | before | after |
| 1 | M | 33 | 1^{st} Dx/1 | 3 | 0 | 31 | 5 | 4.3 | 3.8 |
| 2 | F | 24 | 1^{st} Dx/1 | 7 | 2 | 58 | 9 | 11.6 | 4.3 |
| 3 | M | 33 | Chronic/4 | 5 | 2 | 22 | 4 | 18.3 | 6 |
| 4 | M | 42 | Chronic/2 | 4 | 1 | 32 | 5 | 14 | 6.8 |
| 5 | M | 74 | 1^{st} Dx/1 | 4 | 1 | 22 | 7 | 12.3 | 2.8 |
| 6 | F | 24 | 1^{st} Dx/1 | 2 | 0 | 43 | 11 | 12 | 3.4 |
| 7 | M | 36 | Chronic/2 | 4 | 4 | 28 | 32 | 14 | 3.8 |
| 8 | M | 34 | Chronic/4 | 5 | 1 | 32 | 10 | 20 | 6 |
| 9 | M | 31 | Chronic/3 | 5 | 2 | 43 | 18 | 12 | 6 |
| 10 | F | 34 | 1^{st} Dx/1 | 2 | 0 | 40 | 10 | 14.9 | 4 |
| 11 | M | 38 | 1^{st} Dx/1 | 2 | 0 | 45 | 9 | 12 | 4 |
| 12 | M | 36 | 1^{st} Dx/2 | 2 | 0 | 28 | 7 | 19 | 5 |
| 13 | M | 42 | Chronic/4 | 4 | 1 | 28 | 10 | 18 | 4 |
| 14 | F | 26 | Chronic/2 | 2 | 1 | 20 | 4 | 18 | 3 |
| 15 | M | 33 | Chronic/2 | 4 | 1 | 18 | 10 | 9 | 4 |
| 16 | M | 35 | Chronic/2 | 2 | 0 | 22 | 5 | 14 | 4 |
| 17 | M | 38 | Chronic/4 | 3 | 0 | 34 | 9 | 14 | 8 |
| 18 | M | 42 | 1^{st} Dx/1 | 2 | 1 | 14 | 5 | 18 | 6 |
| 19 | F | 41 | 1^{st} Dx/3 | 3 | 0 | 38 | 11 | 18 | 4 |
| 20 | M | 26 | 1^{st} Dx/4 | 5 | 2 | 48 | 16 | 13 | 6 |
| 20 | 5/15 | 36.1 ± | 10/10 | 3.5 ± | 0.95 ± | 32.3 ± | 9.9 ± | 14.3 ± | 4.7 ± |
| | | 10.6 | | 1.4 | 1.1 | 11.4 | 6.4 | 3.9 | 1.4 |

| | | |
|---|---|---|
| Mean | Mean | Mean |
| decrease = | decrease | decrease = |
| 2.55 cm | 22.4 | 9.6 × 10⁹ L |
| P = | P = | P = |
| 2.893E−009 | 3.713E−008 | 7.162E−010 |

| | | | Weight | | Total | | | |
|---|---|---|---|---|---|---|---|---|
| | Hb | | change | | bilirubin | | ALT | |
| Patient | g/L | | kg | | μmol/L | | IU/L | |
| No. | before | after | before | after | before | after | before | after |
| 1 | 114 | 129 | 57 | 66 | 20 | 10 | 235 | 12 |
| 2 | 101 | 118 | 62 | 75 | 25 | 10 | 162 | 12 |
| 3 | 112 | 125 | 68 | 79 | 18 | 12 | 162 | 12 |
| 4 | 118 | 128 | 74 | 86 | 13 | 15 | 235 | 12 |
| 5 | 120 | 132 | 72 | 82 | 25 | 10 | 162 | 10 |
| 6 | 108 | 115 | 49 | 59 | 20 | 10 | 162 | 12 |
| 7 | 112 | 110 | 62 | 54 | 20 | 20 | 162 | 138 |
| 8 | 108 | 116 | 67 | 74 | 25 | 10 | 162 | 12 |
| 9 | 120 | 132 | 71 | 83 | 25 | 10 | 162 | 12 |
| 10 | 112 | 120 | 47 | 52 | 25 | 10 | 162 | 12 |
| 11 | 118 | 122 | 75 | 83 | 20 | 10 | 235 | 12 |
| 12 | 122 | 128 | 60 | 70 | 25 | 10 | 162 | 12 |
| 13 | 104 | 118 | 66 | 75 | 25 | 10 | 162 | 12 |
| 14 | 118 | 128 | 62 | 67 | 20 | 10 | 132 | 12 |
| 15 | 118 | 120 | 69 | 73 | 20 | 10 | 132 | 12 |
| 16 | 112 | 128 | 71 | 75 | 25 | 10 | 162 | 12 |
| 17 | 122 | 130 | 68 | 77 | 25 | 10 | 162 | 12 |
| 18 | 128 | 132 | 70 | 75 | 25 | 10 | 162 | 12 |
| 19 | 112 | 118 | 59 | 70 | 20 | 10 | 235 | 12 |
| 20 | 102 | 118 | 77 | 85 | 20 | 10 | 132 | 12 |
| 20 | 114 ± | 123.4 ± | 65.3 ± | 73 ± | 22.1 ± | 10.9 ± | 172.1 ± | 18.2 ± |
| | 7.1 | 6.6 | 8.1 | 9.6 | 3.4 | 2.5 | 34 | 28.2 |

| | | | |
|---|---|---|---|
| Mean | Mean | Mean | Mean |
| gain = 9.3 g/L | gain = 7.7 kg | decrease = | decrease = |
| P = | P = | 11.2 μmol/L | 153.9 IU/L |
| 1.419E−007 | 4.604E−007 | P = | P = |
| | | 5.679E−009 | 5.027E−012 |

### Example 6

### Treatment of Multiple Hepatitis Virus Infections Simultaneously

Three patients with dual hepatitis B and C viruses, among whom one with suspected hepatitis delta virus triple infection are given the instant composition. After about one month their liver function tests show normalized function of liver and lack of detectable levels of hepatitis B surface antigen as well as lack of HCV- and HDV-directed antibodies. The viral load test for hepatitis B and C display disappearance of viruses from the blood stream. These results indicate that the immune response resulting from intake of the instant composition is able to clear the virus regardless of the type of virus and whether they are presented alone or in combination. Thus one of embodiments of this invention is a polyvalent vaccine directed against multiple pathogens simultaneously.

### Example 7

### Treatment and Prevention of Enteric Forms of Hepatitis Virus

Two patients are presented in the emergency room of the infectious hospital, one with acute hepatitis A and another with acute hepatitis E. Patients are jaundiced and suffer from severe diarrhea. The stool samples are taken, centrifuged to remove the solid matter, and supernatants are processed to make the composition according to the steps of making said composition. The next day the preparation is administered to bed-ridden patients three times a day (t.i.d.). The fever and comatose status disappeared within one day and one week later the patients are discharged from the hospital with complete recovery and normal lab tests. These results show that the composition is effective even with non-enveloped viruses.

### Example 8

### Use against Unknown Viruses

A patient is brought to the hospital with multiple organs' failure, including liver failure, and life-threatening hemorrhage, necessitating the hospitalization in strictly isolated emergency room. The precise diagnosis could not be made due to negative result from all available tests for known viral infections. Patient is from a Central African country, an area which is known to be endemic for Marburg virus or Ebola virus—members of Filovirus family—belonging to single-stranded RNA viruses with high fatality rate. The blood is taken from the patient and processed to make the instant composition. The next day the composition is administered to the patient intravenously since patient is in coma and not able to swallow the pill. Two days later the patient gained consciousness and is discharged three weeks later due to complete recovery. No definitive diagnosis could be performed although clinical symptoms are highly evocative of hemorrhaging viral infection such as Marburg virus infection, for which there is no proven treatment and outcome is almost always fatal.

### Example 9

### Use against Autoimmune Diseases

A patient with autoimmune hepatitis without any detectable hepatitis virus infection is given the instant composition which is made from non-infected blood from a normal donor. This preparation thus contains an alloantigen only. Within two weeks all signs of autoimmunity cleared and liver function tests are back to normal. The physician decides to give this composition to a patient with multiple sclerosis. Interestingly patient who could not walk prior to treatment, improves dramatically and could now walk without any assistance. Similar outcome is seen in unfertile rheumatic woman with endometriosis and psoriasis who could not conceive for years due to autoimmune rejection of fetus a condition known as unexplained recurrent spontaneous abortions (RSA). After one month on therapy she became pregnant and delivers a healthy baby. Interestingly, due to intake of composition, her endometriosis, psoriasis, rheumatoid arthritis as well as persistent genital Candida albicans disappear without any recurrence.

### Example 10

### Use in Organ Transplant Patients

Two patients in terminal stage of hepatitis with failing liver undergo liver transplant procedure. Instead of being treated with immunosuppressive drugs they opt for receiving the daily dosage of the composition. Patients are alive and doing well without any signs of organ rejection and with normally functioning liver.

All publications, patents, and patent applications cited in this specification are herein incorporated by reference in their entirety as if each individual publication or patent application were specifically and individually indicated to be incorporated by reference.

As will be apparent to those skilled in the art to which the invention pertains, the present invention may be embodied in forms other than those specifically disclosed above, without departing from the spirit or essential characteristics of the invention. The particular embodiments of the invention described above, are, therefore to be considered as illustrative and not restrictive. The scope of the present invention is as set forth in the appended claims rather than being limited to the examples contained in the foregoing description.

What is claimed is:

1. An oral composition comprising a therapeutically effective amount of a partially hydrolyzed antigen, wherein said partially hydrolyzed antigen is from a body fluid of a donor infected with a virus.

2. The composition of claim 1 wherein said virus is a hepatitis virus.

3. The composition of claim 1 wherein said antigen is a hepatitis virus antigen.

4. The composition of claim 1 wherein said antigen is an alloantigen.

5. The composition of claim 1 wherein said antigen is a xenoantigen.

6. The composition of claim 1 wherein said body fluid consists essentially of a whole blood.

7. The composition of claim 1 wherein said body fluid consists essentially of a plasma.

8. The composition of claim 1 wherein said body fluid consists essentially of a serum.

9. The composition of claim 1 wherein said composition is without an immune adjuvant.

10. The composition of claim 1 wherein said composition is a solid composition.

11. The composition of claim 1 wherein said composition is a tablet or pill.

12. The composition of claim 1 wherein said antigen is embedded in a matrix.

13. The composition of claim 12 wherein said matrix is a metal selected from a group of alkali metals, alkaline earth metals, transition metals, metalloids, basic metals, rare earth elements or salts thereof.

14. A method of preventing hepatitis virus infection in a human which comprises administering an effective amount of the composition as claimed in claim 1.

15. A method of treating a human infected with hepatitis virus comprising administering an effective amount of the composition as claimed in claim 1.

16. A process of producing the oral composition of claim 1 useful for treatment or prevention of hepatitis, comprising partially hydrolyzing a hepatitis virus, or a fragment thereof, precipitating with salt of a metal, and formulating into a solid oral composition.

17. The process of producing the oral composition claimed in claim 16 comprising the step of embedding a hepatitis virus or fragment thereof, into salt of a metal.

18. The process of claim 17 wherein said metal is selected from a group essentially consisting of alkali metals, alkaline earth metals, transition metals, metalloids, basic metals, or rare earth elements.

19. The process of claim 17 wherein said salt of metal comprises boride, acetate, phosphate, aluminide, aspartate, benzoate, bromide, carbonate, chloride, chloride hexahydrate, citrate, diboride, diglutamate, diuranate, fluoride, gluconate, hexahydrate, hydride, hydroxide, oxide, iodide, lactate, levulinate, nitrate, nitride, orotate, oxychloride, oxysulfate, perchlorate, peroxide, pidolate, silicide, stearate, sulfate, sulfide, sulfite, or trisilicate salt.

20. The process of claim 17 whereby an additional step of the process comprises heating step.

21. A method for treating a hepatitis, comprising administering to a subject in need thereof, an effective amount of an oral composition, said oral composition comprising a partially hydrolyzed hepatitis virus-infected cell, a hepatitis virus, or fragment thereof.

22. The method of claim 21 said method useful for protection a non-infected subject in need thereof, from a hepatitis virus infection.

23. A vaccine for treatment or prevention of a microbial infection, said vaccine comprising a partially hydrolyzed microbial antigen and an alloantigen in a tablet or pill form.

24. The vaccine of claim 23, said microbial infection is selected from a group essentially consisting of viral infections caused by Hepadnaviridae, Picornaviridae, Flaviridae, Retroviridae, Caliciviridae, Togaviridae, Coronoviridae, Rhabdoviradae, Filoviridae, Paramyxoviridae, Orthomyxoviridae, Bungaviridae, Arenaviridae, Birnaviridae; Parvoviridae, Papovaviridae, Adenoviridae Herpesviridae, Poxyiridae, Iridoviridae, and the like.

25. A composition comprising an partially acid-hydrolyzed hepatitis antigen, an alloantigen, wherein said partially acid-hydrolyzed hepatitis antigen and alloantigen are embedded in a metal salt matrix.

26. The composition of claim 25 wherein said composition is heat-treated.

27. The composition of claim 26 wherein said composition is heat-treated for at least 2 hours.

28. The composition of claim 26 wherein said composition is heat-treated above 56 Centigrades.

29. The composition of claim 26 wherein said composition is heat-treated above 80 Centigrades.

30. The composition of claim 26 wherein said composition is acid-hydrolyzed at pH below 3.

31. The composition of claim 30 wherein said composition is acid-hydrolyzed at pH below 2.

32. A solid composition comprising an acid-hydrolyzed alloantigen embedded in an orally available metal salt matrix.

33. The composition of claim 32 wherein said alloantigen is a human alloantigen.

34. The composition of claim 32 wherein said alloantigen is heat-treated.

35. The composition of claim 32 wherein said metal salt is from a divalent metal.
